(19) United States
(12) Patent Application Publication
(10) Pub. No.: US 2010/0069330 A1
(43) Pub. Date: Mar. 18, 2010

(54) Pharmaceutical Compositions and Methods for Preparing and Using Lipophilic Organosulfur Cell Proliferation Inhibitors

(75) Inventor: Stephen Gately, Scottsdale, AZ (US)

Correspondence Address: DUANE MORRIS LLP - Chicago;IP DEPARTMENT, 190 South LaSalle Street, Suite 3700, CHICAGO, IL, 60603-3433, US

(73) Assignee: Stephen Gately, Scottsdale, AZ (US)

(21) Appl. No.: 12/623,044

(22) Filed: Nov. 20, 2009

Related U.S. Application Data
(63) Continuation of application No. PCT/US08/64370, filed on May 21, 2008.
(60) Provisional application No. 60/939,555, filed on May 22, 2007.

(51) Int. Cl. A61K 31/695; C07F 7/10; A61P 9/00; A61P 19/02; A61P 35/00

(52) U.S. Cl. 514/63; 556/418; 556/419

(57) ABSTRACT

Novel compositions of lipophilic organosulfur compounds, their preparation and use in methods for treating disease are described. Silicon confers lipophilicity that can enhance the penetration of the silicon derivative compounds across the gut wall, cell membranes and blood brain barrier, thus improving therapeutic properties including bioavailability, metabolism, and/or pharmacokinetics. The organosilyl group provides compounds having improved pharmacokinetics. The invention encompasses novel compounds, analogs, prodrugs and pharmaceutically acceptable salts thereof, polymorphic forms, crystalline forms or an amorphous form, pharmaceutical compositions and methods for treatment of diseases, maladies or conditions. Also disclosed are processes for making such compounds as well as intermediates useful in such processes.

The present application claims priority from and is a continuation of PCT/US08/064370 filed May 21, 2008, and claims priority to U.S. application Ser. No. 60/939,555 filed on May 22, 2007, now expired, from which PCT/US08/064,370 also claimed priority. PCT/US08/064370 and U.S. application Ser. No. 60/939,555 are hereby incorporated by reference and entirety.

### BACKGROUND

Cell-permeable small molecules can rapidly perturb the function of their targets and are therefore powerful tools for dissecting dynamic cellular processes. However, such modulators are not available for most of the proteins involved in essential processes, and many of the ones that are available are nonspecific. Many small molecules that specifically affect the mitotic machinery target tubulin, a subunit of the microtubules in the mitotic spindle.

One class of proteins involved in the assembly and maintenance of the mitotic spindle is the family of mitotic kinesins, a subset of the kinesin superfamily. This superfamily contains over 100 proteins, whose other functions include organelle transport and membrane organization. Enzymes in the kinesin superfamily use the free energy of ATP hydrolysis to drive intracellular movement and influence cytoskeleton organization. Kinesins play central roles in mitotic and meiotic spindle formation, chromosome alignment and separation, axonal transport, endocytosis, secretion, and membrane trafficking. The cargo associated with these motor proteins includes intracellular vesicles, organelles, chromosomes, kinetochores, intermediate filaments, microtubules, and even other motors.

Small molecules that conditionally activate or inactivate a protein are valuable tools for analyzing cellular functions of proteins. Their use provides an alternative to conventional biochemical and genetic approaches. However, to date there have been few reports of small molecules that can reversibly alter the function of motor proteins. Butanedione monoxime has been used to probe the role of myosin in cell movement, but its specificity has been questioned. Currently there is a lack of small molecule activators or inhibitors that are specific for one member of the kinesin family. Such an inhibitory molecule with specificity for a particular member of a kinesin class would be useful as an anti-mitotic and also as an anti-cancer, anti-tumorigenic compound.

### SUMMARY OF THE INVENTION

Novel compounds having improved therapeutic properties, including pharmacokinetic properties are disclosed. Methods for preparing and using these compounds are also disclosed. Drugs containing silicon atoms and having beneficial properties are disclosed. A review of silicon chemistry is provided in Tacke and Zilch, Endeavour, New Series, 10, 191-197 (1986); and Showell, G A and Mills, J S, Chemistry challenges in lead optimization: silicon isosteres in drug discovery. Drug Discovery Today 8(12): 551-556, 2003.

Compounds, compositions, methods and systems for inhibiting cell growth are disclosed. Methods, compounds and compositions that are capable of inhibiting mitosis in metabolically active cells are also disclosed.

Methods are also disclosed for administering to a mammal, particularly a human, a treatment-effective amount of a silicon-containing derivative and pharmaceutically acceptable salts thereof.

Compounds that demonstrate enhanced pharmacokinetics, metabolism and/or drug bioavailability are disclosed.

Compounds that demonstrate enhanced lipophilicity, improved gastrointestinal absorption, and enhanced oral bioavailability are also disclosed.

In addition, pharmaceutical compositions containing the disclosed compounds and a pharmaceutically acceptable diluent or carrier are disclosed.

Additional features and advantages of the present invention are described in, and will be apparent from, the following Detailed Description of the Invention.

### DETAILED DESCRIPTION

One embodiment includes a particular class of compounds having one or more silicon atoms. Silicon is lipophilic and thus enhances the penetration of the compounds across the gut wall, cell membranes and blood brain barrier.

The present invention provides compounds that include silicon atom(s) having enhanced pharmaceutical properties.

For example, a compound having general Formula (I) is disclosed.

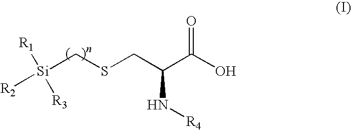

wherein n can be any integral valve that produces an active compound, preferably 1-6;

R₁, R₂, R₃ can be any group that does not substantially interfere with compound formation. Each R can be the same or different and can include, by way of example, methyl, ethyl, n-propyl, iso-propyl, n-butyl, iso-butyl, sec-butyl, tert-butyl, —CH₂CH(CH₂ CH₃)₂, 2-methyl-n-butyl, 6-fluoro-n-hexyl, phenyl, benzyl, cyclohexyl, cyclopentyl, cycloheptyl, allyl, iso-but-2-enyl, 3-methylpentyl, —CH₂-cyclopropyl, —CH₂-cyclohexyl, —CH₂CH₂-cyclopropyl, —CH₂CH₂-cyclohexyl, —CH₂-indol-3-yl, p-(phenyl)phenyl, o-fluorophenyl, m-fluorophenyl, p-fluorophenyl, m-methoxyphenyl, p-methoxyphenyl, phenethyl, benzyl, m-hydroxybenzyl, p-hydroxybenzyl, p-nitrobenzyl, m-trifluoromethylphenyl, p-(CH₃)₂NCH₂CH₂CH₂O-benzyl, p-(CH₃)₃COC(O)CH₂O-benzyl, p-(HOOCCH₂O)-benzyl, 2-aminopyrid-6-yl, p-(N-morpholino-CH₂CH₂O)-benzyl, —CH₂CH₂C(O)NH₂, —CH₂-imidazol-4-yl, —CH₂-(3-tetrahydrofuranyl), —CH₂-thiophen-2-yl, —CH₂ (1-methyl)cyclopropyl, —CH₂-thiophen-3-yl, thiophen-3-yl, thiophen-2-yl, —CH₂—C(O)O-t-butyl, —CH₂—C(CH₃)₃, —CH₂CH(CH₂CH₃)₂, -2-methylcyclopentyl, -cyclohex-2-enyl, —CH[CH(CH₃)₂]COOCH₃, —CH₂CH₂N(CH₃)₂, —CH₂C(CH₃)═CH₂, —CH₂CH═CHCH₃ (cis and trans), —CH₂OH, —CH(OH)CH₃, —CH(O-t-butyl)CH₃, —CH₂OCH₃, —(CH₂)₄NH-Boc, —(CH₂)₄NH₂, —CH₂-pyridyl (e.g., 2-pyridyl, 3-pyridyl and 4-pyridyl), pyridyl (2-pyridyl, 3-pyridyl and 4-pyridyl), —CH₂-naphthyl (e.g., 1-naphthyl and 2-naphthyl), —CH₂—(N-morpholino), p-(N-morpholino-CH₂CH₂O)-benzyl, benzo[b]thiophen-2-yl, 5-chlorobenzo[b]thiophen-2-yl, 4,5,6,7-tetrahydrobenzo[b]thiophen-2-yl, benzo[b]thiophen-3-yl, 5-chlorobenzo[b]thiophen-3-yl, benzo[b]thiophen-5-yl, 6-methoxynaphth-2-yl, —CH₂CH₂S CH₃, thien-2-yl, thien-3-yl, and the like; pharmaceutically acceptable salt thereof, and pharmaceutically acceptable prodrug esters thereof.

R₄ can be H or COCH₃

Another embodiment relates to a pharmaceutical composition comprising a pharmaceutically acceptable excipient and an effective amount of at least one compound of Formula (I). Preferably, the pharmaceutical composition has an excellent pharmacokinetic profile for treating mammals, particularly humans with high safety margin.

Another embodiment relates to processes for producing derivatives of Formula (I) by reacting a compound of Formula (II) with a compound of Formula (III). The reaction product can then be used to generate stable silyl compounds of Formula (I).

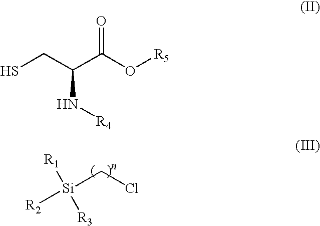

wherein n can be any integral value that produces an active compound, preferably 1-6;

R₁, R₂, R₃ can be any group that does not substantially interfere with compound formation. Each R can be the same or different and can include, by way of example, methyl, ethyl, n-propyl, iso-propyl, n-butyl, iso-butyl, sec-butyl, tert-butyl, —CH₂CH(CH₂CH₃)₂, 2-methyl-n-butyl, 6-fluoro-n-hexyl, phenyl, benzyl, cyclohexyl, cyclopentyl, cycloheptyl, allyl, iso-but-2-enyl, 3-methylpentyl, —CH₂-cyclopropyl, —CH₂-cyclohexyl, —CH₂CH₂-cyclopropyl, —CH₂CH₂-cyclohexyl, —CH₂-indol-3-yl, p-(phenyl)phenyl, o-fluorophenyl, m-fluorophenyl, p-fluorophenyl, m-methoxyphenyl, p-methoxyphenyl, phenethyl, benzyl, m-hydroxybenzyl, p-hydroxybenzyl, p-nitrobenzyl, m-trifluoromethylphenyl, p-(CH₃)₂NCH₂CH₂CH₂O-benzyl, p-(CH₃)₃COC(O)CH₂O-benzyl, p-(HOOCCH₂O)-benzyl, 2-aminopyrid-6-yl, p-(N-morpholino-CH₂CH₂O)-benzyl, —CH₂CH₂C(O)NH₂, —CH₂-imidazol-4-yl, —CH₂-(3-tetrahydrofuranyl), —CH₂-thiophen-2-yl, —CH₂ (1-methyl)cyclopropyl, —CH₂-thiophen-3-yl, thiophen-3-yl, thiophen-2-yl, —CH₂—C(O)O-t-butyl, —CH₂—C(CH₃)₃, —CH₂CH(CH₂CH₃)₂, -2-methylcyclopentyl, -cyclohex-2-enyl, —CH[CH(CH₃)₂]COOCH₃, —CH₂CH₂N(CH₃)₂, —CH₂C(CH₃)═CH₂, —CH₂CH═CHCH₃ (cis and trans), —CH₂OH, —CH(OH)CH₃, —CH(O-t-butyl)CH₃, —CH₂OCH₃, —(CH₂)₄NH-Boc, —(CH₂)₄NH₂, —CH₂-pyridyl (e.g., 2-pyridyl, 3-pyridyl and 4-pyridyl), pyridyl (2-pyridyl, 3-pyridyl and 4-pyridyl), —CH₂-naphthyl (e.g., 1-naphthyl and 2-naphthyl), —CH₂—N-morpholino), p-(N-morpholino-CH₂CH₂O)-benzyl, benzo[b]thiophen-2-yl, 5-chlorobenzo[b]thiophen-2-yl, 4,5,6,7-tetrahydrobenzo[b]thiophen-2-yl, benzo[b]thiophen-3-yl, 5-chlorobenzo[b]thiophen-3-yl, benzo[b]thiophen-5-yl, 6-methoxynaphth-2-yl, —CH₂CH₂SCH₃, thien-2-yl, thien-3-yl, and the like; pharmaceutically acceptable salts thereof, and pharmaceutically acceptable prodrug esters thereof.

R₄ can be H or COCH₃

R₅ can be a carboxylic acid protecting group selected from but not limited to methyl, benzyl, tert-butyl or silyl groups that can form the corresponding esters.

Preferred silicon derivatives of Formula (III) include but are not limited to, chloromethyltrimethylsilane, (chloromethyl)dimethyl(phenyl)silane, (chloromethyl)(methyl)diphenylsilane, (chloromethyl)triphenylsilane, chloropropyltrimethylsilane, (3-chloropropyl)trimethylsilane, (3-chloropropyl)dimethyl(phenyl)silane, (3-chloropropyl)(methyl)diphenylsilane, (3-chloropropyl)triphenylsilane, (4-chlorobutyl)trimethylsilane, (4-chlorobutyl)dimethyl(phenyl)silane, (4-chlorobutyl)(methyl)diphenylsilane, (4-chlorobutyl)triphenylsilane.

Further scope of the applicability of the present invention will become apparent from the detailed description provided below. However, it should be understood that the following detailed description and examples, while indicating certain embodiments of the invention, are given by way of illustration only since various changes and modifications within the spirit and scope of the invention will become apparent to those skilled in the art from this detailed description.

### DEFINITIONS

As discussed above, the present invention provides a novel class of compounds useful for the treatment of cancer and other uncontrolled cell proliferative conditions related thereto. Compounds of this invention comprise those, as set forth herein, and can be grouped into various classes, subgenera and species as disclosed.

“Therapeutically effective”: As used herein, the term “therapeutically effective” is defined as an amount of a compound or composition comprising the compound which is administered to an individual in need thereof to slow or cease uncontrolled or abnormal growth of cells in the individual with sufficiently low toxicity to be useful as a therapeutic.

“Cancer or cancerous growth”: As used herein, the term “cancer” or “cancerous growth” means the uncontrolled, abnormal growth of cells and includes within its scope all the well known diseases that are caused by the uncontrolled and abnormal growth of cells. Non-limiting examples of common cancers include bladder cancer, breast cancer, colon cancer, endometrial cancer, head and neck cancer, lung cancer, melanoma, non-Hodgkin's lymphoma, prostate cancer, and rectal cancer. A complete list of cancers is available from the National Cancer Institute (Bethesda, Md.).

It will be appreciated by one of ordinary skill in the art that asymmetric centers can exist in the compounds of the present invention. Thus, inventive compounds and pharmaceutical compositions thereof may be in the form of an individual enantiomer, diastereomer or geometric isomer, or may be in the form of a mixture of stereoisomers. Additionally, in certain preferred embodiments, as detailed herein, the method of the present invention provides for the stereoselective synthesis of analogues thereof. In addition, where appropriate, steroselective purification methods including crystallization or chromotography are contemplated. Thus, in certain embodiments, the compounds of the invention are enantiopure.

Additionally, the present invention provides pharmaceutically acceptable derivatives of the foregoing compounds.

Methods of treating a subject using these compounds, pharmaceutical compositions thereof, optionally in combination with one or more additional therapeutic agents.

The phrase, “pharmaceutically acceptable derivative”, as used herein, denotes any pharmaceutically acceptable salt, ester, or salt of such ester, of such compound, or any other adduct or derivative which, upon administration to a patient, is capable of providing (directly or indirectly) a compound as otherwise described herein, or a metabolite or residue thereof. Pharmaceutically acceptable derivatives specifically include pro-drugs. A pro-drug is a derivative of a compound, usually with significantly reduced pharmacological activity, which contains an additional moiety that is susceptible to removal in vivo yielding the parent molecule as the pharmacologically active species. An example of a pro-drug is an ester that is cleaved in vivo to yield a compound of interest. Prodrugs of a variety of compounds, and materials and methods for derivatizing the parent compounds to create the pro-drugs, are known and may be adapted to the present invention. Certain exemplary pharmaceutical compositions and pharmaceutically acceptable derivatives will be discussed in more detail herein below.

Certain compounds of the present invention, and definitions of specific functional groups are also described in more detail below. For purposes of this invention, the chemical elements are identified in accordance with the Periodic Table of the Elements, and specific functional groups are defined as described therein.

Furthermore, it will be appreciated by one of ordinary skill in the art that the synthetic methods, as described herein, utilize a variety of protecting groups. By the term “protecting group”, as used herein, it is meant that a particular functional moiety, e.g., O, S, or N, is temporarily blocked so that a reaction can be carried out selectively at another reactive site in a multifunctional compound. In preferred embodiments, a protecting group reacts selectively in good yield to give a protected substrate that is stable to the projected reactions; the protecting group must be selectively removed in good yield by readily available, preferably nontoxic reagents that do not attack the other functional groups; the protecting group forms an easily separable derivative (more preferably without the generation of new stereogenic centers); and the protecting group has a minimum of additional functionality to avoid further sites of reaction. As detailed herein, oxygen, sulfur, nitrogen and carbon protecting groups may be utilized. Exemplary protecting groups are detailed herein, however, it will be appreciated that the present invention is not intended to be limited to these protecting groups; rather, a variety of additional equivalent protecting groups can be readily identified using the above criteria and utilized in the method of the present invention. A variety of carbon protecting groups are described in Myers, A.; Kung, D. W.; Zhong, B.; Movassaghi, M.; Kwon, S. J. Am. Chem. Soc. 1999, 121, 8401-8402, the entire contents of which are hereby incorporated by reference in its entirety.

It will be appreciated that the compounds, as described herein, may be substituted with any number of substituents or functional moieties. In general, the term “substituted” whether preceded by the term “optionally” or not, and substituents contained in formulas of this invention, refer to the replacement of hydrogen radicals in a given structure with the radical of a specified substituent. When more than one position in any given structure may be substituted with more than one substituent selected from a specified group, the substituent may be either the same or different at every position. As used herein, the term “substituted” is contemplated to include all permissible substituents of organic compounds. In a broad aspect, the permissible substituents include acyclic and cyclic, branched and unbranched, carbocyclic. and heterocyclic, aromatic and nonaromatic substituents of organic compounds. For purposes of this invention, heteroatoms such as nitrogen may have hydrogen substituents and/or any permissible substituents of organic compounds described herein which satisfy the valencies of the heteroatoms. Furthermore, this invention is not intended to be limited in any manner by the permissible substituents of organic compounds. Combinations of substituents and variables envisioned by this invention are preferably those that result in the formation of stable compounds useful in the treatment of cancer and/or the inhibition of the growth of or the killing of cancer cells. The term “stable”, as used herein, preferably refers to compounds which possess stability sufficient to allow manufacture and which maintain the integrity of the compound for a sufficient period of time to be useful for the purposes detailed herein.

A compound of the invention may be prepared by any suitable method known in the art. Mixtures of final products or intermediates obtained can be separated on the basis of the physical-chemical differences of the constituents, by known methods, into the pure final products or intermediates, for example by chromatography, distillation, fractional crystallization, or by formation of a salt if appropriate or possible under the circumstances.

Compounds of the invention can be in various crystalline forms and may exist in more than one polymorphic form and as such all forms are intended to be included in the present invention. Amorphous solids, in contrast to crystalline forms, do not possess a distinguishable crystal lattice and do not have an orderly arrangement of structural units. Amorphous forms are generally more soluble, and thus can be desirable for pharmaceutical purposes because the bioavailability of amorphous compounds may be greater than their crystalline counterparts. Certain methods for generating these forms are known and it is within the skill of one having skill in the art to produce them using such art. Mixtures of amorphous and crystalline forms are also contemplated.

The preparation of the compounds of Formula (I) are affected by a variety of procedures depending primarily upon the specific definitions of the n and R₁, R₂, R₃, R₄, moieties. One of skill can appreciate that chemical reactions and procedures analogous to those known in the art and the selection of a particular route to obtain particular compounds is governed by known principles and can be obtained using methods that are analogous to the disclosed methods.

Preferred silicon derivatives that can be generated using such methods include the following:
(R)-2-amino-3-((trimethylsilyl)methylthio)propanoic acid
(R)-2-amino-3-((dimethyl(phenyl)silyl)methylthio)propanoic acid
(R)-2-amino-3-((methyldiphenylsilyl)methylthio)propanoic acid
(R)-2-amino-3-((triphenylsilyl)methylthio)propanoic acid
(R)-2-acetamido-3-((trimethylsilyl)methylthio)propanoic acid
(R)-2-acetamido-3-((dimethyl(phenyl)silyl)methylthio)propanoic acid
(R)-2-acetamido-3-((methyldiphenylsilyl)methylthio)propanoic acid
(R)-2-acetamido-3-((triphenylsilyl)methylthio)propanoic acid
(R)-2-amino-3-((trimethylsilyl)propylthio)propanoic acid
(R)-2-amino-3-((dimethyl(phenyl)silyl)propylthio)propanoic acid
(R)-2-amino-3-((methyldiphenylsilyl)propylthio)propanoic acid
(R)-2-amino-3-((triphenylsilyl)propylthio)propanoic acid
(R)-2-acetamido-3-((trimethylsilyl)propylthio)propanoic acid
(R)-2-acetamido-3-((dimethyl(phenyl)silyl)propylthio)propanoic acid
(R)-2-acetamido-3-((methyldiphenylsilyl)propylthio)propanoic acid
(R)-2-acetamido-3-((triphenylsilyl)propylthio)propanoic acid
(R)-2-amino-3-((trimethylsilyl)butylthio)propanoic acid
(R)-2-amino-3-((dimethyl(phenyl)silyl)butylthio)propanoic acid
(R)-2-amino-3-((methyldiphenylsilyl)butylthio)propanoic acid
(R)-2-amino-3-((triphenylsilyl)butylthio)propanoic acid
(R)-2-acetamido-3-((trimethylsilyl)butylthio)propanoic acid
(R)-2-acetamido-3-((dimethyl(phenyl)silyl)butylthio)propanoic acid
(R)-2-acetamido-3-((methyldiphenylsilyl)butylthio)propanoic acid
(R)-2-acetamido-3-((triphenylsilyl)butylthio)propanoic acid

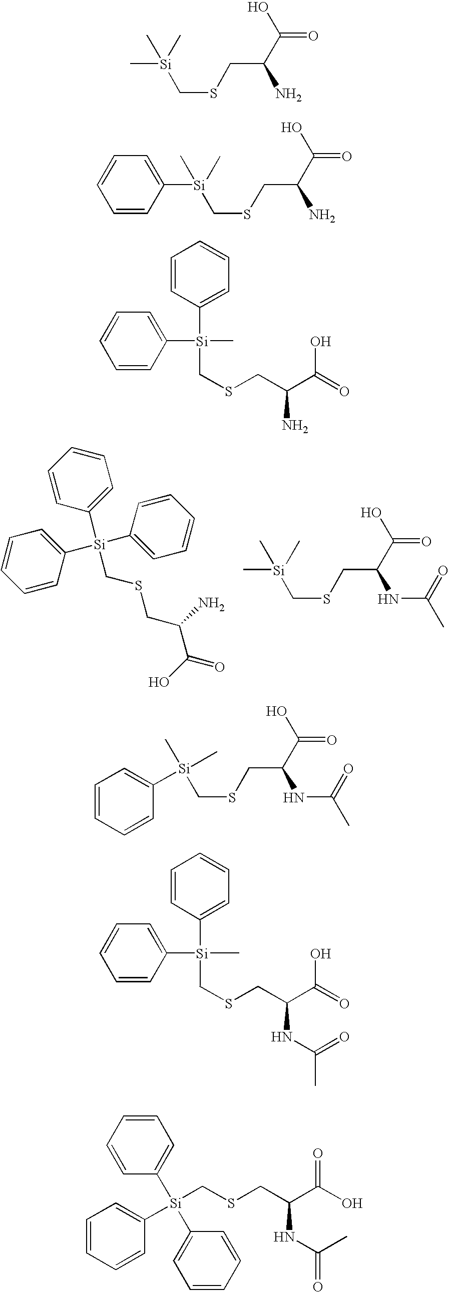

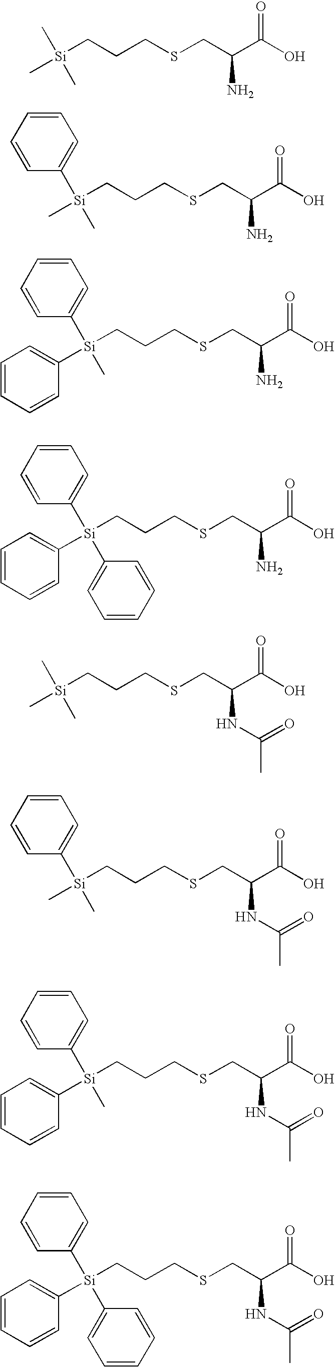

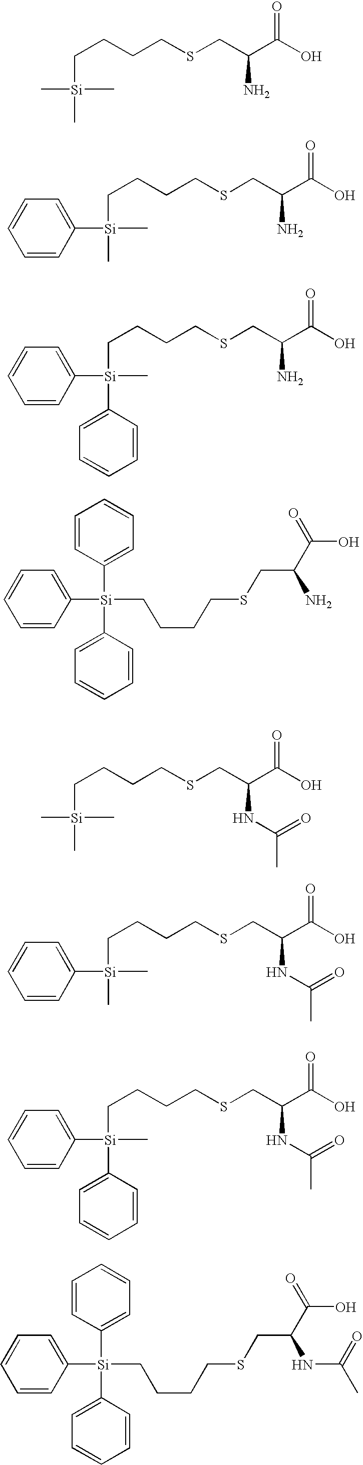

Treatment is contemplated in mammals, particularly humans, as well as those mammals of economic or social importance, or of an endangered status. Examples may be livestock or other animals expressly for human consumption, or domesticated animals such as dogs, cats, or horses. Also contemplated is the treatment of birds and poultry, such as turkeys, chickens, and fowl of the like.

Determination of a therapeutically effective amount is well within the capability of those skilled in the art, especially in light of the detailed disclosure provided herein.

Toxicity and therapeutic efficacy of the compounds described herein can be determined by standard pharmaceutical procedures in cell cultures or experimental animals, e.g., by determining the IC₅₀ and the LD₅₀ (lethal dose causing death in 50% of the tested animals) for a subject compound. The data obtained from these cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition.

Depending on the severity and responsiveness of the condition to be treated, dosing can also be a single administration of a slow release composition, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

The present invention provides compounds, compositions, methods and systems for inhibiting cell growth. More specifically, the present invention provides for methods, compounds and compositions that are capable of inhibiting mitosis in metabolically active cells.

The disclosed compounds can be used to treat any cell system where the control of cellular growth and cell division is desired. One non-limiting example of an application of a compound of this invention is the use as an anti-mitotic anti-cancer drug. Other examples include controlling cell division of the immune system in diseases such as rheumatoid arthritis or endometriosis.

In a preferred embodiment, a method of treating an individual with uncontrolled or abnormal cell growth is provided. Certain disclosed compositions can be used to treat individuals with cells that have become cancerous tumors. Generally, abnormal cell growth is associated with cancer. However, other diseases resulting from uncontrolled cell growth (e.g. cardiovascular diseases, rheumatoid arthritis etc.) may be treated with compositions and methods of the present invention.

### Pharmaceutical Compositions

The present invention provides novel compounds having unexpected antitumor and anti-cell proliferative activity, and thus the inventive compounds are useful for the treatment of cancer. Accordingly, in another aspect of the present invention, pharmaceutical compositions are provided, wherein these compositions comprise any one of the compounds as described herein, and optionally comprise a pharmaceutically acceptable carrier.

In certain preferred embodiments, these compositions optionally further comprise one or more additional therapeutic agents. In certain other embodiments, the additional therapeutic agent is an anticancer agent, as discussed in more detail herein. It will also be appreciated that certain of the compounds of present invention can exist in free form for treatment, or where appropriate, as a pharmaceutically acceptable derivative thereof. According to the present invention, a pharmaceutically acceptable derivative includes, but is not limited to, pharmaceutically acceptable salts, esters, salts of such esters, or any other adduct or derivative which upon administration to a patient in need is capable of providing, directly or indirectly, a compound as otherwise described herein, or a metabolite or residue thereof, e.g., a prodrug.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describe pharmaceutically acceptable salts in detail in J. Pharmaceutical Sciences, 66: 1-19 (1977), incorporated herein by reference. The salts can be prepared in situ during the final isolation and purification of the compounds of the invention, or separately by reacting the free base function with a suitable organic acid. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hernisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Additionally, as used herein, the term “pharmaceutically acceptable ester” refers to esters that hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include formates, acetates, propionates, butyrates, acrylates and ethylsuccinates.

Furthermore, the term “pharmaceutically acceptable prodrugs” as used herein refers to those prodrugs of the compounds of the present invention which are, within the scope of sound medical judgment, suitable for use in contact with the tissues of humans and lower animals with undue toxicity, irritation, allergic response, and the like, commensurate with a reasonable benefit/risk ratio, and effective for their intended use, as well as the zwitterionic forms, where possible, of the compounds of the invention. The term “prodrug” refers to compounds that are rapidly transformed in vivo to yield the parent compound of the above formula, for example by hydrolysis in blood.

As described above, the pharmaceutical compositions of the present invention additionally comprise a pharmaceutically acceptable carrier, which, as used herein, includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, 18th Edition, Part 8 discloses various carriers used in formulating pharmaceutical compositions and known techniques for the preparation thereof. Except insofar as any conventional carrier medium is incompatible with the anti-viral compounds of the invention, such as by producing any undesirable biological effect or otherwise interacting in a deleterious manner with any other component(s) of the pharmaceutical composition, its use is contemplated to be within the scope of this invention. Some examples of materials which can serve as pharmaceutically acceptable carriers include, but are not limited to, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

In yet another aspect, according to the methods of treatment of the present invention, tumor cells are killed, or their growth is inhibited by contacting said tumor cells with an inventive compound or composition, as described herein. Thus, in still another aspect of the invention, a method for the treatment of cancer is provided comprising administering a therapeutically effective amount of an inventive compound, or a pharmaceutical composition comprising an inventive compound to a subject in need thereof, in such amounts and for such time as is necessary to achieve the desired result.

In certain embodiments of the present invention a “therapeutically effective amount” of the inventive compound or pharmaceutical composition is that amount effective for killing or inhibiting the growth of tumor cells. The compounds and compositions, according to the method of the present invention, may be administered using any amount and any route of administration effective for killing or inhibiting the growth of tumor cells. Thus, the expression “amount effective to kill or inhibit the growth of tumor cells”, as used herein, refers to a sufficient amount of agent to kill or inhibit the growth of tumor cells. The exact amount required will vary from subject to subject, depending on the species, age, and general condition of the subject, the severity of the infection, the particular anticancer agent, its mode of administration, and the like.

The anticancer compounds of the invention are preferably formulated in dosage unit form for ease of administration and uniformity of dosage. The expression “dosage unit form” as used herein refers to a physically discrete unit of anticancer agent appropriate for the patient to be treated. It will be understood, however, that the total daily usage of the compounds and compositions of the present invention will be decided by the attending physician within the scope of sound medical judgment. The specific therapeutically effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

Furthermore, after formulation with an appropriate pharmaceutically acceptable carrier in a desired dosage, the pharmaceutical compositions of this invention can be administered to humans and other animals orally, rectally, parenterally, intracisternally, intravaginally, intraperitoneally, topically (as by powders, ointments, or drops), bucally, as an oral or nasal spray, or the like, depending on the severity of the infection being treated. In certain embodiments, the compounds of the invention may be administered orally or parenterally one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution that, in turn, may depend upon crystal size and crystalline form. Alternatively, dissolving or suspending the drug in an oil vehicle accomplishes delayed absorption of a parenterally administered drug form. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar—agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and- bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in microencapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, eardrops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

As discussed above, the compounds of the present invention are useful as anticancer agents, and thus may be useful in the treatment of cancer, by effecting tumor cell death or inhibiting the growth of tumor cells. In general, the inventive anticancer agents are useful in the treatment of cancers and other proliferative disorders, including, but not limited to adrenal cortical cancer, breast cancer, cervical cancer, colon and rectal cancer, gastric cancer, leukemia, lung cancer, melanoma, multiple myeloma, non-Hodgkin's lymphoma, ovarian cancer, pancreatic cancer, prostate cancer, and, thymoma to name a few. In certain embodiments, the inventive anticancer agents are active against leukemia cells and melanoma cells, and thus are useful for the treatment of leukemias (e.g., myeloid, lymphocytic, myelocytic and lymphoblastic leukemias) and malignant melanomas. In still other embodiments, the inventive anticancer agents are active against solid tumors and also kill and/or inhibit the growth of multidrug resistant cells (MDR cells).

Certain compounds could be used for the treatment of angiogenesis-related disorders in a subject. In a method the compounds can be administered to a subject in need of angiogenesis inhibition. The compounds could be used in the treatment of neoplasia, including metastasis; opthalmo logical conditions such as corneal graft rejection, ocular neovascularization, retinal neovascularization including neovascularization following injury or infection, diabetic retinopathy, macular degeneration, retrolental fibroplasia and neovascular glaucoma; ulcerative diseases such as gastric ulcer; pathological, but non-malignant, conditions such as hemangiomas, including infantile hemaginomas, angiofibroma of the nasopharynx and avascular necrosis of bone; and disorders of the female reproductive system such as endometriosis.

It will also be appreciated that the compounds and pharmaceutical compositions of the present invention can be employed in combination therapies, that is, the compounds and pharmaceutical compositions can be administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, an inventive compound may be administered concurrently with another anticancer-agent), or they may achieve different effects (e.g., control of any adverse effects).

For example, other therapies or anticancer agents that may be used in combination with the inventive anticancer agents of the present invention include surgery, radiotherapy (in but a few examples, y-radiation, neutron beam radiotherapy, electron beam radiotherapy, proton therapy, brachytherapy, and systemic radioactive isotopes, to name a few), endocrine therapy, biologic response modifiers (interferons, interleukins, and tumor necrosis factor (TNF) to name a few), hyperthermia and cryotherapy, agents to attenuate any adverse effects (e.g., antiemetics), and other approved chemotherapeutic drugs, including, but not limited to, alkylating drugs (mechlorethamine, chlorambucil, Cyclophosphamide, Melphalan, Ifosfamide), antimetabolites (Methotrexate), purine antagonists and pyrimidine antagonists (6-Mercaptopurine, 5-Fluorouracil, Cytarabile, Gemcitabine), spindle poisons (Vinblastine, Vincristine, Vinorelbine, Paclitaxel), podophyllotoxins (Etoposide, Irinotecan, Topotecan), antibiotics (Doxorubicin, Bleomycin, Mitomycin), nitrosoureas (Carmustine, Lomustine), inorganic ions (Cisplatin, Carboplatin), enzymes (Asparaginase), and hormones (Tamoxifen, Leuprolide, Flutamide, and Megestrol), to name a few. More comprehensive discussion of updated cancer therapies can be found on internet websites run by the National Cancer Institution, the FDA and others.

In still another aspect, the present invention also provides a pharmaceutical pack or kit comprising one or more containers filled with one or more of the ingredients of the pharmaceutical compositions of the invention, and in certain embodiments, includes an additional approved therapeutic agent for use as a combination therapy. Optionally associated with such container(s) can be a notice in the form prescribed by a governmental agency regulating the manufacture, use or sale of pharmaceutical products, which notice reflects approval by the agency of manufacture, use or sale for human administration.

The invention is claimed as follows:

1. A compound of Formula I 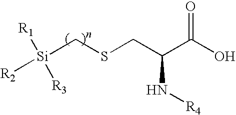
wherein n is an integer;
and wherein R₁, R₂, R₃ can be the same or different and can be selected from the chemical groups consisting of methyl, ethyl, n-propyl, iso-propyl, n-butyl, iso-butyl, sec-butyl, tert-butyl, —CH₂CH(CH₂CH₃)₂, 2-methyl-n-butyl, 6-fluoro-n-hexyl, phenyl, benzyl, cyclohexyl, cyclopentyl, cycloheptyl, allyl, iso-but-2-enyl, 3-methylpentyl, —CH₂-cyclopropyl, —CH₂-cyclohexyl, —CH₂CH₂-cyclopropyl, —CH₂CH₂-cyclohexyl, —CH₂-indol-3-yl, p-(phenyl)phenyl, o-fluorophenyl, m-fluorophenyl, p-fluorophenyl, m-methoxyphenyl, p-methoxyphenyl, phenethyl, benzyl, m-hydroxybenzyl, p-hydroxybenzyl, p-nitrobenzyl, m-trifluoromethylphenyl, p-(CH₃)₂NCH₂CH₂CH₂O-benzyl, p-(CH₃)₃COC(O)CH₂O-benzyl, p-(HOOCCH₂O)-benzyl, 2-aminopyrid-6-yl, p-(N-morpholino-CH₂CH₂O)-benzyl, —CH₂CH₂C(O)NH₂, —CH₂-imidazol-4-yl, —CH₂-(3-tetrahydrofuranyl), —CH₂-thiophen-2-yl, —CH₂ (1-methyl)cyclopropyl, —CH₂-thiophen-3-yl, thiophen-3-yl, thiophen-2-yl, —CH₂—C(O)O-t-butyl, —CH₂—C(CH₃)₃, —CH₂CH(CH₂CH₃)₂, -2-methylcyclopentyl, -cyclohex-2-enyl, —CH[CH(CH₃)₂]COOCH₃, —CH₂CH₂N(CH₃)₂, —CH₂C(CH₃)═CH₂, —CH₂CH═CHCH₃ (cis and trans), —CH₂OH, —CH(OH)CH₃, —CH(O-t-butyl)CH₃, —CH₂OCH₃, —(CH₂)₄NH-Boc, —(CH₂)₄NH₂, —CH₂-pyridyl (e.g., 2-pyridyl, 3-pyridyl and 4-pyridyl), pyridyl (2-pyridyl, 3-pyridyl and 4-pyridyl), —CH₂-naphthyl (e.g., 1-naphthyl and 2-naphthyl), —CH₂—(N-morpholino), p-(N-morpholino-CH₂CH₂O)-benzyl, benzo[b]thiophen-2-yl, 5-chlorobenzo[b]thiophen-2-yl, 4,5,6,7-tetrahydrobenzo[b]thiophen-2-yl, benzo[b]thiophen-3-yl, 5-chlorobenzo[b]thiophen-3-yl, benzo[b]thiophen-5-yl, 6-methoxynaphth-2-yl, —CH₂CH₂ SCH₃, thien-2-yl, thien-3-yl, and the like; pharmaceutically acceptable salt thereof, and pharmaceutically acceptable prodrug esters thereof and wherein R₄ is H or COCH₃.

2. The compound according to claim 1, selected from a group consisting of:
(R)-2-amino-3-((trimethylsilyl)methylthio)propanoic acid
(R)-2-amino-3-((dimethyl(phenyl)silyl)methylthio)propanoic acid
(R)-2-amino-3-((methyldiphenylsilyl)methylthio)propanoic acid
(R)-2-amino-3-((triphenylsilyl)methylthio)propanoic acid
(R)-2-acetamido-3-((trimethylsilyl)methylthio)propanoic acid
(R)-2-acetamido-3-((dimethyl(phenyl)silyl)methylthio)propanoic acid
(R)-2-acetamido-3-((methyldiphenylsilyl)methylthio)propanoic acid
(R)-2-acetamido-3-((triphenylsilyl)methylthio)propanoic acid
(R)-2-amino-3-((trimethylsilyl)propylthio)propanoic acid
(R)-2-amino-3-((dimethyl(phenyl)silyl)propylthio)propanoic acid
(R)-2-amino-3-((methyldiphenylsilyl)propylthio)propanoic acid
(R)-2-amino-3-((triphenylsilyl)propylthio)propanoic acid
(R)-2-acetamido-3-((trimethylsilyl)propylthio)propanoic acid
(R)-2-acetamido-3-((dimethyl(phenyl)silyl)propylthio)propanoic acid
(R)-2-acetamido-3-((methyldiphenylsilyl)propylthio)propanoic acid
(R)-2-acetamido-3-((triphenylsilyl)propylthio)propanoic acid
(R)-2-amino-3-((trimethylsilyl)butylthio)propanoic acid
(R)-2-amino-3-((dimethyl(phenyl)silyl)butylthio)propanoic acid
(R)-2-amino-3-((methyldiphenylsilyl)butylthio)propanoic acid
(R)-2-amino-3-((triphenylsilyl)butylthio)propanoic acid
(R)-2-acetamido-3-((trimethylsilyl)butylthio)propanoic acid
(R)-2-acetamido-3-((dimethyl(phenyl)silyl)butylthio)propanoic acid
(R)-2-acetamido-3-((methyldiphenylsilyl)butylthio)propanoic acid
(R)-2-acetamido-3-((triphenylsilyl)butylthio)propanoic acid.

3. The compound of claim 1 wherein n is 1-6.

4. The compound of Formula (I) further comprising an inert carrier wherein the compound and inert carrier are contained in a pharmaceutical composition.

5. The pharmaceutical composition of claim 4, further comprising a second distinct active agent.

6. The compound of claim 1, wherein the compound is a diasteriomer, racemate, single enantiomer.

7. The compound of claim 1, wherein the compound is in a hydrated form, solvated form, polymorphic form, crystalline form or an amorphous form.

8. A method for the preparation of a compound of Formula I according to claim 1 which comprises:
(a) reacting a compound of Formula II with a compound of Formula III 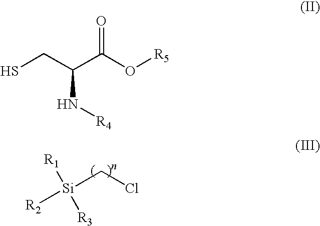
wherein n is an integer; and wherein R₁, R₂, R₃ can be the same or different and can include a group selected from the chemical groups consisting of methyl, ethyl, n-propyl, iso-propyl, n-butyl, iso-butyl, sec-butyl, tert-butyl, —CH₂CH(CH₂CH₃)₂, 2-methyl-n-butyl, 6-fluoro-n-hexyl, phenyl, benzyl, cyclohexyl, cyclopentyl, cycloheptyl, allyl, iso-but-2-enyl, 3-methylpentyl, —CH₂-cyclopropyl, —CH₂-cyclohexyl, —CH₂CH₂-cyclopropyl, —CH₂CH₂-cyclohexyl, —CH₂-indol-3-yl, p-(phenyl)phenyl, o-fluorophenyl, m-fluorophenyl, p-fluorophenyl, m-methoxyphenyl, p-methoxyphenyl, phenethyl, benzyl, m-hydroxybenzyl, p-hydroxybenzyl, p-nitrobenzyl, m-trifluoromethylphenyl, p-(CH₃)₂NCH₂CH₂CH₂O-benzyl, p-(CH₃)₃COC(O)CH₂O-benzyl, p-(HOOCCH₂O)-benzyl, 2-aminopyrid-6-yl, p-(N-morpholino-CH₂CH₂O)-benzyl, —CH₂CH₂C(O)NH₂, —CH₂-imidazol-4-yl, —CH₂-(3-tetrahydrofuranyl), —CH₂-thiophen-2-yl, —CH₂ (1-methyl)cyclopropyl, —CH₂-thiophen-3-yl, thiophen-3-yl, thiophen-2-yl, —CH₂—C(O)O-t-butyl, —CH₂—C(CH₃)₃, —CH₂CH(CH₂CH₃)₂, -2-methylcyclopentyl, -cyclohex-2-enyl, —CH[CH(CH₃)₂]COOCH₃, —CH₂CH₂N(CH₃)₂, —CH₂C(CH₃)═CH₂, —CH₂CH═CHCH₃ (cis and trans), —CH₂OH, —CH(OH)CH₃, —CH(O-t-butyl)CH₃, —CH₂OCH₃, —(CH₂)₄NH-Boc, —(CH₂)₄NH₂, —CH₂-pyridyl (e.g., 2-pyridyl, 3-pyridyl and 4-pyridyl), pyridyl (2-pyridyl, 3-pyridyl and 4-pyridyl), —CH₂-naphthyl (e.g., 1-naphthyl and 2-naphthyl), —CH₂—(N-morpholino), p-(N-morpholino-CH₂CH₂O)-benzyl, benzo[b]thiophen-2-yl, 5-chlorobenzo[b]thiophen-2-yl, 4,5,6,7-tetrahydrobenzo[b]thiophen-2-yl, benzo[b]thiophen-3-yl, 5-chlorobenzo[b]thiophen-3-yl, benzo[b]thiophen-5-yl, 6-methoxynaphth-2-yl, —CH₂CH₂ SCH₃, thien-2-yl, thien-3-yl, and the like; pharmaceutically acceptable salt thereof, and pharmaceutically acceptable prodrug esters thereof; and wherein R₄ is H or COCH₃ and R₅ is a carboxylic acid protecting group selected from but not limited to methyl esters, benzyl esters, tert-butyl esters or silyl esters.

9. A method for treatment of a subject with uncontrolled or abnormal cell growth comprising administering to the subject a therapeutically effective amount of a compound of Formula (I) or a pharmaceutically acceptable salt, prodrug ester, hydrated form, solvated form, polymorphic form, crystalline form or an amorphous form thereof.

10. The method of claim 9, in which the condition or disorder is angiogenesis.

11. The method of claim 9, in which the condition or disorder is cancer.

12. The method of claim 9, in which the condition is rheumatoid arthritis.

13. The method of claim 9, in which the condition is cardiovascular disease.

14. The method of claim 9, wherein the subject is a mammal.

15. The method of claim 9, wherein the subject is human.

16. The method of claim 9, wherein the compound is administered in a pharmaceutical composition that includes a pharmaceutically acceptable carrier.

17. The method of claim 9, wherein said administering effect is by intranasal, transdermal, intradermal, oral, buccal, parenteral, topical, rectal, or inhalation administration.

18. The method of claim 9, wherein the composition further includes a formulating agent selected from the group consisting of a suspending agent, a stabilizing agent and a dispersing agent.

19. The method of claim 9, wherein a therapeutically effective amount of a compound of claim 1 or a pharmaceutically acceptable salt, hydrated form, solvated form, polymorphic form, crystalline form or an amorphous form thereof is administered in advance of or concurrently with an anti-neoplastic compound selected from the group consisting of antibiotic agents, alkylating agents, antimetabolite agents, hormonal agents, immunological agents, interferon agents, wherein: the amount of conjunctive therapy and the amount of the compound of the invention together comprise a neoplasia-treating-effective amount; and the neoplasia is sensitive to such treatment.

20. The method for treatment of a subject with uncontrolled or abnormal cell growth of claim 9 in a subject in need of such treatment wherein a therapeutically effective amount of a compound of Formula (I) or a pharmaceutically acceptable salt, hydrated form, solvated form, polymorphic form, crystalline form or an amorphous form thereof is administered with ionizing radiation: the amount of radiation and the amount of the compound of Formula (I) together comprise a treatment-effective amount; such that cell growth is slowed.
